(19) United States
(12) Patent Application Publication
(10) Pub. No.: US 2020/0163326 A1
(43) Pub. Date: May 28, 2020

(54) CRYOPRESERVATION

(71) Applicant: UCL BUSINESS LTD, London, Greater London (GB)

(72) Inventors: PAOLO DE COPPI, London, Greater London (GB); Luca URBANI, London, Greater London (GB); Massimo PINZANI, London, Greater London (GB); Guiseppe MAZZA, London, Greater London (GB); Claire CROWLEY, London, Greater London (GB)

(73) Assignee: UCL BUSINESS LTD, London, Greater London (GB)

(21) Appl. No.: 16/617,310

(22) PCT Filed: Jun. 1, 2018
(86) PCT No.: PCT/EP2018/064475

§ 371 Date: Nov. 26, 2019

(30) Foreign Application Priority Data
Jun. 1, 2017 (GB) 1708729.7

(51) Int. Cl. A01N 1/02; A61L 27/38; A61K 35/545

(52) U.S. Cl. CPC A01N 1/0221; A61L 27/3804; A01N 1/0284; A61K 35/545; A61L 2430/28; A61L 27/3873; A61L 27/3882; A61L 2430/30; A61L 2430/22; A01N 1/0231

(57) ABSTRACT

Methods and materials for the cryopreservation of cellularised scaffolds used for therapeutic or pharmacological testing purposes that provide a cultured scaffold on which cells have been seeded, equilibrate the cellularised scaffold with a cryopreservative composition comprising culture medium and between 5 and 30% of a cryoprotectant such as DMSO, freeze the equilibrated cellularised scaffold by reducing the temperature continuously by about −1° C./minute to about −80° C., and store the frozen cellularised scaffold at a temperature of between −135° C. and −198° C.

### TECHNICAL FIELD

The present invention relates generally to methods and materials for use in the cryopreservation of cellularised scaffolds.

### BACKGROUND ART

Tissue engineering (TE) is proving to be a viable and important alternative to conventional treatment of damaged or diseased organs and tissues [1-3]. A variety of engineered organs and tissues are currently in preclinical trials (trachea, heart valves, larynx, blood vessels, bladder) [4,5].

A particularly promising TE approach is the use of decellularisation to create non-immunogenic matrices which are then recellularised with autologous or otherwise compatible cells. The process of decellularisation removes the cellular compartment of tissues and organs using detergents and enzymes. Importantly, the extracellular matrix of the scaffold is preserved, thus maintaining the original architecture and composition of the tissue [3,4,6-8], but avoiding any potential immunorejection [9]. The donor tissue does not need to be of human origin, but can be harvested from an anatomically matched species [10], thus potentially solving the significant donor organ shortage problem.

Examples of publications concerning tissue engineering decellularised matrices include those relating to the intestine [11,12] the oesophagus [7], the lung [14], and the diaphragm [15]. In one example, a tissue engineered trachea was prepared using autologous stem cells and transplanted into a child [5]. Later reports show that the trachea was still integrated and furthermore the engineered organ had grown with the child, illustrating the great utility of TE [16].

WO201742232 describes improved methods for the production of implants, particularly luminal tissue implants, where the implants are engineered by seeding of an acellular scaffold or matrix with muscle cell precursors and fibroblasts, for example, injection seeding using particular ratios of cells.

As tissue engineering applications are increasingly utilised in the clinic, a major limiting factor is the ability to provide sufficient numbers of suitable cellularised scaffolds promptly when required. The ability to provide such engineered organs “off the shelf” on demand could significantly increase the number of patients who could benefit from this treatment.

In this respect it would be desirable to be able to pre-prepare recellularised scaffolds and store them in a way which would substantially preserve the structure and function of the construct.

Methods have been proposed in the art for the cryopreservation of decellularised scaffolds or of native tissues. Examples include: Franchini et al, Blood Transfus., 2009, 7, 100-105; Bonenfant et al, Biomaterials, 2013, 34, 3231-3245; Gallo et al, Heart Vessels, 2016: 1862-1873; Brockbank et al, Cell Tissue Banks, 2012, 13, 663-671; Poornejad et al, Organogenesis, 2015, 11, 30-45.

However the structure and properties of these scaffolds or tissues is not equivalent to that of cellularised scaffolds, and therefore methods which may be applicable to these cell-free scaffolds or native tissues cannot be reasonably expected to apply to cellularised scaffolds.

Chen et al (Biomaterials, 2011, 32, 8426-8435) investigated the cryopreservation of epithelial sheets in the presence of trehalose. Chitosan-gelatin membranes seeded with keratinocytes were frozen using different cryopreservation solutions (including trehalose, and one excluding trehalose) by keeping the samples at 4° C. for 30 minutes, followed by placement into liquid nitrogen. Cell suspensions were cryopreserved by step freezing (4° C. for 30 min, −20° C. for 2 hours, −80° C. overnight and then liquid nitrogen).

Costa et al (Tissue Engineering, 2012, 18, 852-858) investigated the cryopreservation of porous scaffolds which were fiber meshes based on a starch and poly(caprolactone) blend and seeded with goat bone marrow stem cells. The authors apparently used cryopreservation directly into liquid nitrogen at −196° C.

U.S. Pat. No. 6,638,709 B2 relates to cryopreserved composite living constructs (CCLCs) which are comprised of separated layers of cultured fibroblasts and cultured keratinocytes and to processes for making CCLCs. The CCLCs are prepared by equilibrating with cryoprotectant solutions based on a non-cell-penetrating component and a cell-penetrating component, freezing, and storing at cryogenic temperatures. Prior to use, they are thawed and rinsed to substantially remove the cryoprotectants. The freezing is carried out by a specified temperature lowering program which uses specific varying temperature lowering rates and holding phases.

U.S. Pat. No. 8,367,059 B2 concerns cryopreserved bone constructs. In one embodiment, porous hydroxyapatite-chitosan-gelatin (HCG) scaffolds are provided in a perfusion bioreactor, cells are then seeded in the HCG scaffolds in the perfusion bioreactor, cell culture media is perfused through and the bioreactor operated so as to allow for cell seeding and growth in the HCG scaffold. Subsequently the HCG-cell constructs are perfused with a suitable cryopreservation fluid and then cooled in a specific step-wise manner.

Nevertheless, it can be seen from the foregoing that novel methods for the successful long-term storage of engineered organs and the like by cryopreservation would provide a useful contribution to the art.

### GENERAL DISCLOSURE OF THE INVENTION

The present invention provides a particular “slow cooling” medium and method for cellularised scaffolds which has been shown to maintain cellular function and integrity of the scaffold post-thawing in a number of different types of cellularised scaffold. In preferred embodiments the invention utilises components which have already been GMP approved for clinical use, and provides a sterile and relatively inexpensive methodology for preserving materials.

The method of the invention has been successfully used for the cryopreservation of scaffolds such as oesophagus and liver engineered constructs. By way of example, thawing of the recellularised liver showed that the cells are capable of producing albumin to a comparable level to cells in a recellularised scaffold that was maintained in culture.

This finding is particularly surprising since other slow cooling methods used in the cryopreservation of a decellularised scaffold and of a native tissue have been reported to be introduce injuries into the materials (see Gallo et al & Brockbank et al supra.)

The provision of a novel and effective system for the cryopreservation of TE organs opens up new clinical possibilities in this field.

In various aspects the invention provides methods and materials for the cryopreservation of cellularised scaffolds, which may be used for therapeutic or pharmacological testing purposes, which methods comprise: (i) providing a cultured scaffold on which cells have been seeded; (ii) equilibrating said cellularised scaffold with a cryopreservative composition comprising culture medium and between 5 and 30% of a cryoprotectant such as DMSO; (iii) freezing the equilibrated cellularised scaffold by reducing the temperature at a defined rate to a defined temperature; (iv) storing the frozen cellularised scaffold at a temperature of between −135° C. and −198° C.

### DETAILED DISCLOSURE OF THE INVENTION

Thus in a first aspect, there is provided a method for cryopreservation of a cellularised scaffold, which method comprises:

(i) providing a cellularised scaffold;

(ii) equilibrating said cellularised scaffold with a cryopreservative composition comprising culture medium and between 5 and 30% of a cryoprotectant;

(iii) freezing the equilibrated cellularised scaffold by reducing the temperature at between −0.5° C. and −2° C. (e.g. between −0.8° C. and −1.2° C. e.g. about −1° C.)/minute to between −75° C. and −85° C. (e.g. between −78° C. to −82° C. e.g. about −80° C.);

(iv) storing the frozen cellularised scaffold at a temperature of between −135° C. and −198° C., e.g. about −160° C.

In the “slow cooling” methods of the invention step (iii) is preferably carried out continuously, and not in a step-wise manner.

By “cellularised scaffold” is meant an acellular scaffold which has been cell seeded and cultured. In a particular embodiment, the cellularised scaffold may be a recellularised scaffold (i.e. cell seeded scaffolds from a decellularised tissue). Examples of recellularised scaffolds and other cellularised scaffolds (i.e. utilising artificial or synthetic scaffolds) are described hereinafter.

Sources of acellular scaffolds or matrices are well known in the art. For example, WO0214480 refers to five general categories of scaffold in the art: (1) non-degradable synthetic polymers; (2) degradable synthetic polymers; (3) non-human collagen gels, which are non-porous; (4) non-human collagen meshes, which are processed to a desired porosity; and (5) human (cadaveric) decellularized collagenous tissue.

An “acellular” scaffold typically does not comprise cells or cellular components. However, it will be appreciated that, for example, where a scaffold is used from a biological source, e.g. a decellularised scaffold, it is possible that some cells may remain on the scaffold e.g. after decellularisation, as discussed below.

In one embodiment herein the scaffold is an artificial or a synthetic polymer scaffold. Examples of synthetic polymers include Dacron and Teflon which may be processed into a variety of fibres and weaves. Other polymers used as synthetic tissue matrices include polygalactide and polydioxanone.

Other synthetic scaffolds may be proteinaceous in nature, e.g. primarily consist of purified proteins such as collagen.

Non-synthetic scaffolds may also be proteinaceous in nature, or primarily consist of a collagenous extracellular matrix (ECM).

Preferably the scaffold will be a decellularized (biological) matrix. The scaffold may be xenogeneic, i.e. it originates from or is derived from a donor of a different species than the recipient, for example, a human recipient. Alternatively, the scaffold may be allogeneic.

In this connection, substrates suitable for decellularization are, inter alia, decellularized animal-derived scaffolds e.g. porcine-derived, rat-derived or rabbit-derived.

Any known decellularization method can be employed to provide the scaffold. In general decellularization methods employ a variety of chemical, biochemical, and/or physical means to disrupt, degrade, and/or destroy cellular components and/or modify the matrix in which the cells are embedded so as to facilitate removal of the cells and cellular components, typically leaving an ECM scaffold. The terms “scaffold” and “matrix” are used interchangeably herein, unless context demands otherwise. WO0214480 (supra) describes methods of decellularizing native tissues, including inter alia any of a variety of detergents, emulsification agents, proteases, and/or high or low ionic strength solutions, known in the art. The invention encompasses the use of decellularized scaffolds produced by any decellularization technique that removes a substantial fraction of the cells while leaving the matrix substantially intact.

The removal of “a substantial fraction” of cells may typically refer to the removal of at least 50%, for example, at least 60, 70, 80, 90, 95 or 99% of cells and particularly the removal of all, or virtually all, the cells. Reference to leaving the matrix “substantially intact” refers to retaining the presence of at least 40, 50, 60, 70, 80, 90, 95 or 99% of the matrix e.g. of the ECM.

Therefore, optionally, step (i) of the method of the first aspect comprises:

(ia) providing an acellular scaffold;

(ib) seeding the acellular scaffold with the cells;

(ic) culturing the seeded scaffold to produce said cellularised scaffold.

Optionally step (ia) comprises:

(ia-1) providing a tissue or organ or sample thereof;

(ia-2) decellularizing said tissue or organ or sample thereof using one or both of detergents and enzymes to provide an acellular scaffold.

Alternatively step (ia) comprises:

(ia-1) providing an artificial or synthetic acellular scaffold.

Culture Media

For seeding purposes the cells may be delivered in a suitable medium such as those well known in the art. Examples include supplemented MEGACELL medium (5% FBS; 1% Penicillin Streptomycin; 1% L-Glutamine; 1% non-essential amino acids; 0.1 mM Beta Mercapto Ethanol; 5 ng/ml Basic FGF), Dulbecco's Modified Eagle's Medium (DMEM), etc., or gels such as Matrigel etc. The medium may contain collagen, fibronectin, or the like. Suitable examples of media appropriate to cellularised scaffolds are described in the publications referred to herein, and the Examples below.

The culture or growth media is mixed with between 5 and 30% of a cryoprotectant prior to cryopreservation. For example in one embodiment the final cryopreservative composition comprises: 50% Fetal Bovine Serum (FBS); 40% MEGACELL Medium with supplements and 10% DMSO.

Seeding and Culture Conditions

Seeding and culture conditions for providing a cellularised scaffold are known in the art and described in the publications referred to herein.

It will be appreciated that the number of cells seeded onto a scaffold will depend on several factors, including the size of the scaffold, the density of cells required on the scaffold, the time period for which the scaffold will be cultured after seeding, and the use of the scaffold. Thus, it may not be necessary for cells to be seeded across the whole scaffold, e.g. if a subsequent culture step is to be carried out. Particularly, however, cells may be seeded to cover at least 10, 20, 30, 40, 50, 60, 70, 80 or 90% of the scaffold. It will further be appreciated that one of more surfaces of a scaffold may be seeded with cells, depending, for example, on the eventual use of the cellularised scaffold.

Any type of cells may be used to produce a cellularised scaffold as defined herein, from any source. For example, the cells may be fibroblasts, mesoangioblasts, epithelial cells etc. As discussed below, where a cellularised scaffold forms a construct which is suitable for transplantation into a subject to repair or replace a damaged organ or tissue, the cells seeded on the scaffold typically may correspond to those present in the organ or tissue which is to be repaired or replaced. The source of such cells is discussed below.

Typically culture of the seeded construct will be in a “bioreactor”.

As discussed, for example in US2014/0341862, reactors suitable for a very wide variety of different tissue constructs are known in the prior art. Particularly suitable for tubular constructs is, for example, a reactor as depicted in DE 199 15 610 (Bader), or one as described in EP 0 320 441 (Sulzer). An (e.g.) tubular vessel may be clamped in such a reactor and thus subjected to through-flow of medium or blood, as comes closest to the subsequent natural situation of integration in the body.

This bioreactor may incorporate a removable cassette which can be transferred from a decellularization bioreactor, subjected to seeding, and then introduced to a recellularisation bioreactor (see e.g. WO2017042232).

Therapeutically Relevant Constructs

Thus, in preferred embodiments, the invention concerns the production and cryopreservation of constructs which mimic a tissue or organ that needs to be repaired or replaced. In this case, the scaffold is seeded with cells, which can populate the scaffold, resulting in an artificial construct which can be transplanted into a subject. Thus, a cellularised scaffold may form a construct for tissue or organ repair.

The cells used in such methods will typically be autologous, i.e. originate from or are derived from the intended recipient of the tissue or organ construct. However, cells for use in the method may also be allogeneic, i.e. obtained or derived from a subject who is not the recipient of the tissue or organ construct to be generated. Further, xenogeneic cells may be used, i.e. cells derived from a different species to the recipient of the tissue/organ construct.

The terms “tissue” or “organ” are used interchangeably herein with respect to the construct, unless context demands otherwise.

The term “subject” or “patient” as used herein refers to any mammal, e.g. a domestic animal such as a dog, cat etc., an agricultural animal, such as a horse, pig or cow etc., or a human. In one embodiment the subject or patient may be a neonate or infant, particularly a human neonate or infant.

The general strategy for producing replacement tissues utilizes mammalian cells that are seeded onto an appropriate scaffold for cell culture. The cells can be obtained from the intended recipient (e.g., from a biopsy), in which case they are often expanded in culture before being used to seed the scaffold. Cells can also be obtained from other sources (e.g., established cell lines). After seeding, cell growth is generally continued in the laboratory and/or in the patient following implantation of the engineered tissue (e.g. comprising or consisting of a cellularised scaffold).

Pharmaceutical Testing

In other embodiments the cellularised scaffolds of the invention may be utilised in pharmacological research. Examples include cellularised scaffolds based on the scaffolds described in WO2017017474, which describe the production of (inter alia) decellularised tissue scaffold consisting of acellular extracellular matrix (ECM) from the source tissue which retains the three dimensional architecture, ECM composition and bioactivity of the ECM of the source tissue. These can be repopulated with cells suitable for the research of assays in hand. Examples of the constructs described in WO2017017474 include liver tissue cubes.

These are and other aspects and embodiments of the invention will now be described in more detail:

In one embodiment the construct is a luminal tissue implant.

Reference to a “luminal” construct, or the like, refers to a construct which is suitable for replacement of, or implantation into, a luminal organ or tissue, such as those described below, rather than strictly the structure of the construct itself. For example the construct may simply be in the form of a sheet, which can be used or applied as desired. Reference to tissue constructs should be understood accordingly. Thus reference to an oesophageal construct refers to a construct which is suitable for implantation into the oesophagus, or as an oesophageal replacement, and a bowel construct refers to a construct which is suitable for implantation into the bowel, or as a bowel replacement. In one embodiment, the construct may have a luminal or tubular shape

In one embodiment the scaffold is itself tubular.

In one embodiment the scaffold is derived from a luminal organ which has been decellularized.

In one embodiment the scaffold is of non-human origin.

In one embodiment the cellularised scaffold is a luminal tissue implant, which has been engineered by seeding of an acellular scaffold or matrix with muscle cell precursors and fibroblasts as described in WO2017042232. Thus the cells may be a combination of mesoangioblasts and fibroblasts seeded into and/or onto the matrix, wherein said mesoangioblasts and fibroblasts are seeded separately, simultaneously or sequentially. The scaffold may also be seeded with neural crest cells e.g. of mouse origin.

In one embodiment the culture medium is an FBS/Megacell medium.

Thus in one embodiment the methods of the invention comprise:

(i) providing a scaffold;

(ii) seeding a combination of mesoangioblasts and fibroblasts (and optionally further cells e.g. neural crest cells) into and/or onto the matrix of the scaffold, wherein said mesoangioblasts and fibroblasts are seeded separately, simultaneously or sequentially;

(iii) culturing the seeded scaffold to produce an implantable construct;

(iv) cryopreserving the construct as described herein.

In one embodiment the cellularised scaffold is a tissue engineered oesophagus, which may optionally be one suitable for a neonate or infant.

By way of non-limiting example, a typical oesophageal construct suitable for a neonate may be around 8-10 mm across and 4-5 cm long when in the relaxed state,

In one embodiment the cellularised scaffold is a decellularised oesophagus seeded with mesoangioblasts (e.g. human) and fibroblasts (e.g. mouse or human).

In one embodiment, the scaffold is derived from a solid organ which has been decellularized. The scaffold may be any 3-dimensional solid e.g. one which is actually or approximately: spherical, cuboid, cylindrical, hexagonal prismatic, conical, frustoconical, pyramidal and so on.

In one embodiment, the cellularised scaffold is tissue engineered liver, for example as described in WO2017017474 or WO2015185912.

In one embodiment the cellularised scaffold is a decellularised liver tissue seeded with human hepatic cells e.g. stem cells, iPS cells, or a human hepatic cell line, which is optionally the HepG2 cell line.

In one embodiment, the scaffold is a hydrogel scaffold, such as that derived from human liver ECM scaffold as described in WO2015185912 A1

In one embodiment, the cellularised scaffold is a hydrogel scaffold seeded with a human hepatic cell line, which is optionally the HepG2 cell line.

In one embodiment the culture medium is an FBS/alpha MEM medium.

In one embodiment the cellularised scaffold is a model tissue (e.g. liver model tissue) for pharmacological research or therapeutic purpose. Such scaffolds may be shaped solids as described above, with maximum dimensions of between 3 mm to 30 mm.

In one embodiment the cellularised scaffold is cuboid having e.g. around 3, 4, 5, 6, 7, 8, 9, 10 mm side dimensions.

In other embodiments the cellularised scaffold may be selected from tissue engineered lung, intestine, pancreas muscle or bladder.

Such cellularised scaffolds may be used for therapeutic purposes or pharmacological research.

Cryopreservative Medium

In some embodiments the cryopreservative composition comprises 80% or more culture medium. For example, the cryopreservative composition may comprise less than 20%, preferably between 5% and 15%, more preferably 8 to 12%, more preferably about 10%, cryoprotectant.

The cryoprotectant may be selected from any one of more from the list consisting of: dimethyl sulfoxide (DMSO); ethylene glycol; glycerol; 2-Methyl-2,4-pentanediol; propylene glycol; sucrose; trehalose.

Cryopreservation Conditions

In one embodiment step (ii) is carried out below ambient temperature, for example at a temperature of less than 20, 15, 10 or 5° C., optionally at about 0 to 4° C.

This is followed by step (iii) which comprises freezing the equilibrated cellularised scaffold by reducing the temperature at between −0.5° C. and −2° C./minute to between −75° C. and −85° C. A preferred rate of cooling is about −1° C./minute i.e. −0.8, 0.9, 1.1, or 1.2° C./min.

This cooling step is preferably continued to about −80° C. e.g. −78, −79, −81, −82° C.

The cooling in step (iii) may be achieved by placing the equilibrated cellularised scaffold within one or more containers at around −80° C.

The subsequent cooling in step (iv) may be achieved by placing the equilibrated cellularised scaffold within one or more containers in the vapour phase of liquid nitrogen. This will achieve a temperature of about −160° C.

Preferably the cooling step or steps are continuous i.e. do not comprise a step-wise temperature reduction whereby the cellularised scaffold is removed and returned to the cooling environment.

In some embodiments step (iv) is carried out for at least 1, 2, 4, or 4 weeks, or more. For example, step (iv) may be carried out for at least 12, 16, 20, 24 weeks.

Once it is desired to utilise the frozen cellularised scaffold, this can conveniently be achieved by thawing the frozen cellularised scaffold rapidly in a 37° C. water bath. This may be carried out either at the storage location, or point of care or use.

Cell Viability

In one embodiment, the thawed cellularised scaffold will have a cell viability expressed as the percent of total number of viable cells present in the cellularised scaffold of at least about 70% of the original number of viable cells originally present in the uncryopreserved cellularised scaffold.

In one embodiment, the thawed cellularised scaffold retain at least about 50% of the original number of cells present in the uncryopreserved cellularised scaffold.

In one embodiment, the metabolic activity of viable cells is at least 50% of the original metabolic activity of the viable cells originally present in the cellularised scaffold.

In one embodiment, the scaffold structural integrity is substantially unaffected by the cryopreservation.

Methods for quantifying and assessing cell viability, cell number and metabolic activity, as well as scaffold integrity, are described hereinafter. Examples of the methods of analysis for cell viability used include histological analysis, bioluminescence imaging and quantification of albumin.

Other Aspects and Utilities

In one aspect, the invention provides a cryopreserved cellularised scaffold obtained according to the methods described herein, and use of the same in a method of treatment or surgery.

In one aspect, the invention provides a kit comprising a cryopreserved cellularised scaffold of the invention and one or more containers.

Optionally the kit further comprises instructions or labeling for the use of said kit for therapeutic or pharmacological research purposes.

Specifically, kits can optionally comprise instructions or labeling that describes how to maintain, store, thaw, and/or use the cryopreserved cells and constructs. Kits can also optionally comprise media for storage, maintenance, thawing, and/or growth of the cryopreserved cellularised scaffolds.

One aspect of the invention provides a novel treatment for patients with chronic illnesses in which an organ replacement may ultimately be required if the patient's condition degenerates substantially. This can undesirably result in an emergency situation where there is limited time available to prepare a treatment, but where a tissue engineered construct or organ replacement may take a number of months to prepare.

By use of the present invention, such materials can be prepared in advance and cryopreserved until they are required.

Thus the invention provides a method of treatment of a subject with a chronic illness leading to organ failure, which method comprises:

(i) providing a cellularised scaffold which is a tissue engineered organ replacement using autologous cells of the subject;

(ii) cryopreserving the organ replacement according to the methods described herein;

(iii) thawing the organ replacement when it is required by said subject;

(iv) treating said subject using said organ replacement.

Induced Pluripotent Stem (iPS) cells are a promising source of cells in tissue engineering. For example it has been reported that a bank of 100 iPS cells created using “universal donor” could be used for 40% of the population.

This makes it feasible for recellularised organ replacements to be prepared and cryopreserved pre-emptively in a variety of sizes suitable for providing replacements that are suitable for a large proportion of the population on demand, without the need to harvest and expand autologous cells. Such allogeneic materials could be provided at short notice to the point of care.

Thus the invention provides a system for providing allogeneic tissue engineered organ replacements, which system comprises:

(i) providing a plurality cellularised scaffolds which are allogeneic tissue engineered organ replacements using universal donor iPS cells;

(ii) cryopreserving the organ replacements according to the methods described herein;

(iii) identifying a subject in need to an organ replacement;

(iv) identifying a compatible cryopreserved allogeneic tissue engineered organ replacement;

(v) thawing the organ replacement when it is required by said subject;

(vi) treating said subject using said organ replacement.

Optionally the cellularised scaffold or cellularised scaffolds are preserved remotely from point of care of the subject.

In respect of each of the possible therapeutic or surgical methods, uses, and utilities practised on the human or animal body described herein, there is also provided: use of the materials (cellularised scaffolds) in the preparation of a medicament or implant for use in that therapeutic or surgical context, and the material (cellularised scaffolds) for use as a medicament or implant in that therapeutic or surgical context.

Engineered micro-scaffolds have great utility in drug testing, since their three-dimensional structure provides a more realistic model than the use of two-dimensional cell cultures.

An example system is based on human liver ECM hydrogel and liver micro-scaffolds (see e.g. MuBbach, Franziska, et al. “Bioengineered Livers: A New Tool for Drug Testing and a Promising Solution to Meet the Growing Demand for Donor Organs.” European Surgical Research 57.3-4 (2016): 224-239). Examples are also provided in WO2017017474.

Such systems are typically prepared on demand for testing or implantation, and shipped rapidly in ideal physiological conditions. Cryopreservation according to the present invention allows for production in advance.

Thus the invention provides a method for providing a three dimensional engineered micro-scaffold for use in pharmacological research purposes, which method comprises:

(i) providing a cellularised scaffold which is a three dimensional engineered micro-scaffold suitable for pharmacological research purposes;

(ii) cryopreserving the engineered micro-scaffold according to the methods described herein;

(iii) thawing the micro-scaffold when it is required.

Optionally the method further comprises utilising the thawed scaffold as desired e.g. by contacting it with a putative pharmacological agent, determining a physiological or other parameter of the thawed scaffold, comparing that parameter to a corresponding scaffold not contacted with the putative agent, and so on.

Throughout this specification, including the claims which follow, unless the context requires otherwise, the word “comprise” and variations such as “comprises” and “comprising,” will be understood to imply the inclusion of a stated integer or step or group of integers or steps but not the exclusion of any other integer or step or group of integers or steps.

Where a material described herein is disclosed as comprising or including a specified mixture of components, it will be understood that there is likewise disclosed mutatis mutandis a material “consisting” of those components, or “consisting essentially” of those components.

It must be noted that, as used in the specification and the appended claims, the singular forms “a,” “an,” and “the” include plural referents unless the context clearly dictates otherwise. Thus, for example, reference to “a drying step” includes combinations of two or more such drying steps, and the like.

Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

This disclosure includes information that may be useful in understanding the present invention. It is not an admission that any of the information provided herein is prior art or relevant to the presently claimed invention, or that any publication specifically or implicitly referenced is prior art

Any headings and sub-titles herein are included for convenience only, and are not to be construed as limiting the disclosure in any way.

The invention will now be further described with reference to the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### FIGURES

FIG. 1-FIG. 2C: Cryopreservation of a recellularised tissue engineered oesophagus.

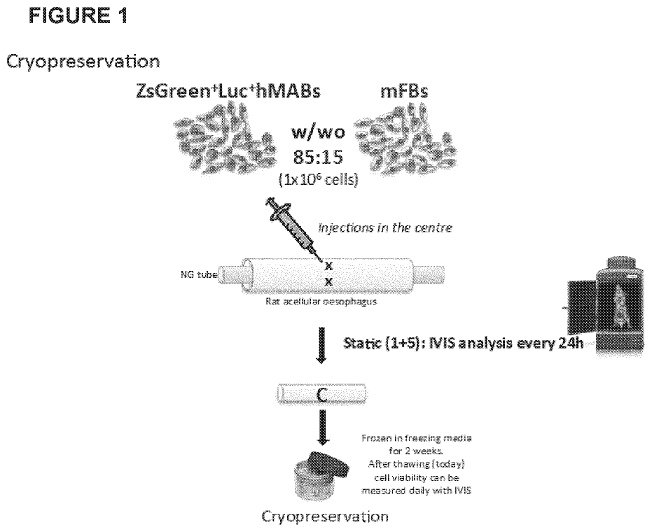

Rat decellularised oesophagi were seeded with Luc⁺Zs-Green⁺ human mesoangioblasts and mouse fibroblasts and mouse GFP⁺ neural crest cells, cultured in a bioreactor for up to 11 days then cryopreserved with the developed protocol for two weeks, thawed with developed protocol and cultured for a further 14 days.

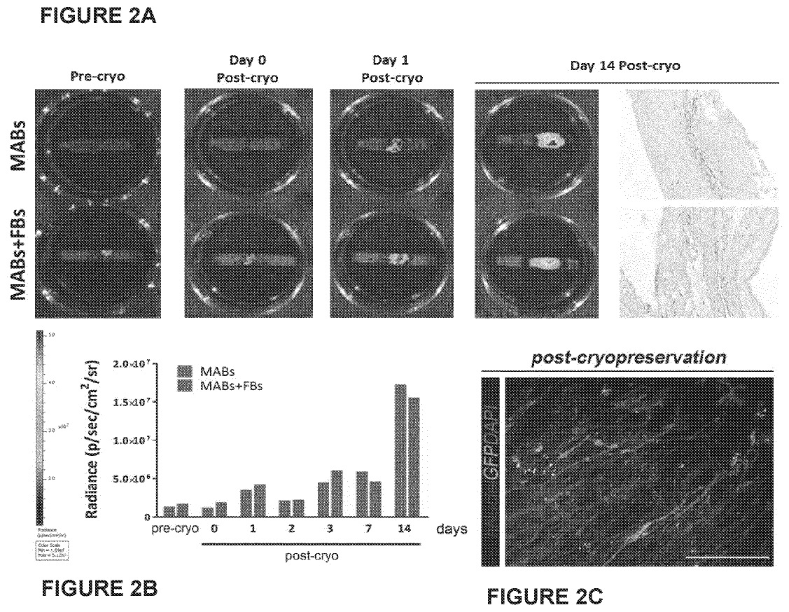

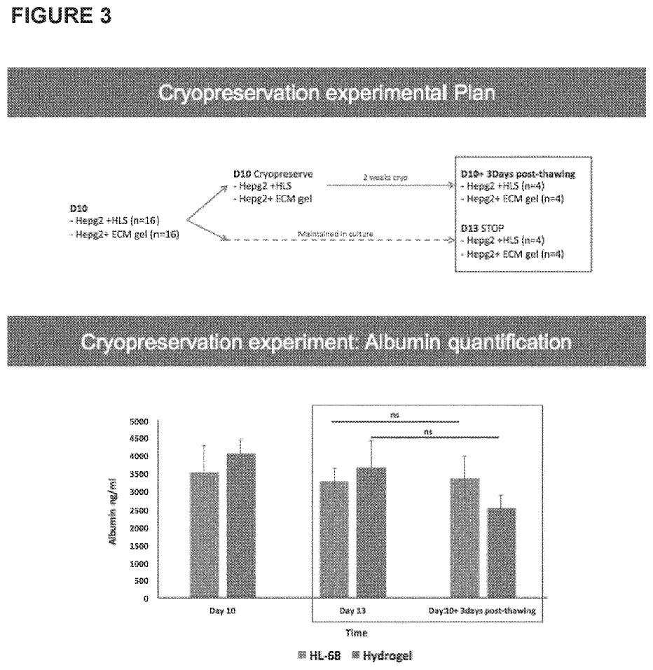

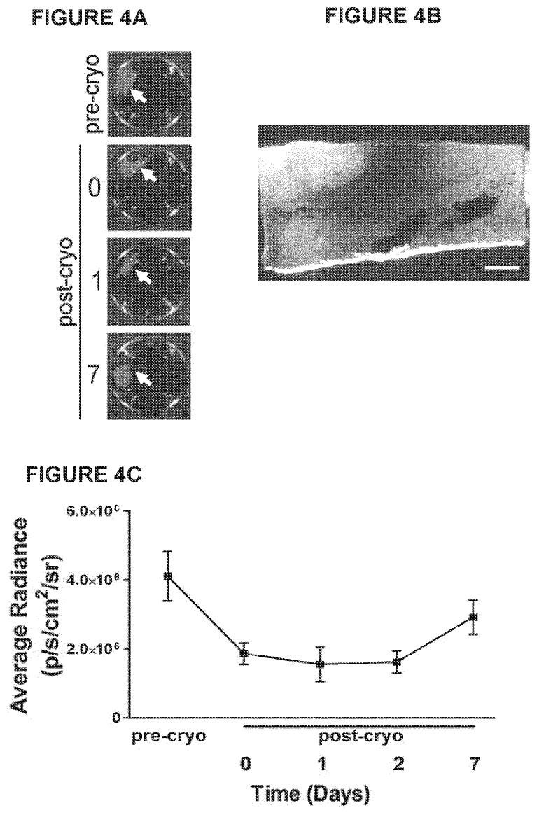

The cells were tracked using bioluminescence imaging with an In Vivo Imagine System (IVIS, Perkin Elmer) pre and post cryopreservation. H&E staining shows the cells in the scaffold 14 days post-cryo (FIG. 2A, FIG. 2B). In FIG. 2B, the results for the ‘MABs’ are shown on the left and the ‘MABs+FBs’ on the right for each time point.

In FIG. 2C a representative section of the cryo-preserved oesophagus containing human mesangioblasts and fibroblasts and mouse GFP+ neurocrest cells.

FIG. 3. Cryopreservation of recellularised human liver scaffolds and liver hydrogel.

0.5M HepG2 were seeded on 16 hydrogels and 16 HL-68. Half of each scaffold type were cryopreserved for 2 weeks using developed method. Post-cryopreservation, there was no significant different between the fresh scaffolds and the thawed cryopreserved scaffolds. In the histogram, the results for HL-68 are shown on the left and the results for hydrogel shown on the right for each time point.

FIG. 4A-FIG. 4C: Maintenance of cell viability after storage

Oesophageal scaffolds were seeded with Luc⁺ZsGreen⁺hMAB+mFB and were cultured in static conditions and then cryopreserved for 2 weeks. The results demonstrate that the bio-engineered muscle cryopreserved with a slow-cooling process show maintenance of cell viability after storage.

In FIG. 4A bioluminescence images of a representative scaffold seeded with Luc+ZsGreen+hMAB+mFB and cultured in static for 8 days (pre-cryo) and for further 7 days after 2 weeks of cryopreservation are shown. Locations of detected bioluminescence radiance are indicated with an arrow.

In FIG. 4B a representative image of MTT colorimetric assay performed on a seeded-scaffold following cryopreservation. Scale bar: 1 mm.

FIG. 4C shows average radiance detected using bioluminescence imaging before and after cryopreservation at different time points. Data: mean±SEM (n=3).

### EXAMPLES

### Example 1—Materials and Methods

Confirming Scaffold Integrity

Histology

Samples are fixed for 24 hours in 10% neutral buffered formalin solution in PBS (pH 7.4; Sigma, UK) at RT, washed in dH₂O, dehydrated in graded alcohol, embedded in paraffin and sectioned at 5 μm. Tissue slides are stained with haematoxylin and eosin (H&E; Leica, Germany).

DNA Quantification

DNA is isolated using a tissue DNA isolation kit following the manufacturer's instructions (PureLink Genomic DNA MiniKit, Invitrogen, UK).

ECM Component Quantification

Collagen, elastin and glycosaminoglycan (GAG) content can be quantified using the total collagen assay kit (Biocolor, UK), the FASTIN elastin assay and the GAG assay kit (Biocolor, UK) respectively—see [14]

Biomechanical Testing

To evaluate the biomechanical properties of oesophagi, specimens can be tested and subjected to uniaxial longitudinal tension until failure [12]. Uniaxial tension may be applied using an Instron 5565, with specimens in the form of flat dumbbells (20 mm) loaded at a constant tension rate of 100 mm/min. The thickness of the samples can be measured using a digital electronic micrometer (RS components, US) at three places of the dumbbell and averaged.

Scanning Electron Microscopy (SEM)

Samples are fixed in 2.5% glutaraldehyde (Sigma, UK) in 0.1 M phosphate buffer and left for 24 hrs at 4° C. SEM may be performed as described in [14].

Confirming Cell Viability, Number and Metabolic Activity

Cell viability may be carried out according to methods well known in the art, for example as described in U.S. Pat. No. 6,638,709. These include assessing “construct cell density”, the total number of viable cells per unit area; “cell viability”, the percent of the total number of cells that are viable; and “metabolic activity”, a measure of the overall vigor of the viable cells in terms of their ability to metabolize nutrients and perform other cell maintenance functions. Additional measurements include histologic examination of the cellularised construct for the presence, configuration, and distribution of cells within and on the construct.

Briefly, cell number and cell viability can be measured by releasing cells from the construct and determining cell viability and cell number by a Hemocytometer using Trypan Blue dye exclusion to differentiate living from dead cells.

Metabolic Activity may be measured using samples incubated with Alamar Blue dye. The assay measures mitochondrial activity using a non-cytotoxic Alamar Blue dye which diffuses into the cell mitochondria and undergoes a reduction-oxidation reaction to give a fluorescent product that is read by a fluorescent spectrophotometer. Metabolic Activity may be measured using an MTT assay. The yellow MTT (3-(4, 5-dimethylthiazolyl-2)-2, 5-diphenyltetrazolium bromide) is reduced by metabolically active cells by the action of dehydrogenase enzymes to generate reducing equivalents such as NADH and NADPH, resulting in a purple formazan that can be solubilised and quantified by spectrophotometric means. In this way, the MTT assay can be used to measure cell viability.

Cell viability can also be measured using an assay that detects markers of apoptosis, for example, a caspase-3 assay. An exemplary caspase-3 assay is described in the examples.

Histology requires a visual assessment of the structure and morphology of the construct and cells therein (see Examples herein).

IVIS Imaging:

Lentivirus Production

The lentiviral transfer vector pHIV-LUC-ZsGreen (used in FIG. 2A) was a gift from Dr. Bryan Welm (Department of Surgery, University of Utah, purchased through Addgene Inc. MA, USA, Plasmid #39196) and was used to generate a lentivirus containing both ZsGreen florescent protein and firefly luciferase from an EF1-alpha promoter. This third generation lentivirus required the packaging plasmids pRSV-Rev (Addgene Plasmid #12253) and pMDLg/pRRE (Addgene #12251) as well as the VSV-G envelope plasmid pMD2.G. (Addgene Plasmid #12259).

Briefly, lentiviral vectors were produced by co-transfecting 293T cells with the above plasmids. Transfection of plasmids was in 293T cells using a jetPEI/plasmid mix according to manufacturer's instructions. After 6 hours at 37° C., the medium (DMEM containing 10% FBS; Gibco, U.K.) was exchanged for virus collection. After 24 hours, this virus-containing medium was purified by centrifugation at 2500 rpm (4° C.) and filtered through a 0.45 μm membrane. Medium was ultracentrifuged at 50,000 g for 2 hours at 4° C. (SW28 rotor, Optima LE80K Ultracentrifuge, Beckham, High Wycombe, UK). The viral pellet was re-suspended in 100 μl pre-cooled serum-free DMEM (Gibco, U.K.) and the virus was stored at −80° C. until use.

Viral titres were calculated by transduction efficacy in HeLa cells, a cell line known to be permissive to viral transduction. HeLa cells were expanded in complete DMEM. Cells were seeded at 5×104 cells per well in a 24-well plate. A dilution series (1:5) from 20 μl/ml virus to 0.0032 μl/ml virus was created in a total volume of 500 μl per well. Cells were cultured overnight and changed for fresh medium the following day. Transduction efficacy was determined by flow cytometric analysis of the proportion of cells expressing the fluorescent protein ZsGreen 72 hours after transfection. Viral titres were calculated with the following formula.

Viral titre (iu/ml)=Number of cells seeded×percentage of florescent positive cells/Volume of virus (ml)

Viral titres were calculated from volume of virus used to transduce cells at 15-25% transduction efficacy.

Lentiviral Transduction of Stromal Cells and FACS Sorting.

Stromal cells derived from muscle were transduced with the lentivirus as described above but scaled to T25 flasks and tested at increasing MOI. Transduction efficacy was determined by FACs as a percentage of cells transduced. In order to obtain a pure population of transduced cells, cells were FACS sorted following expansion of cells by one passage. Briefly cells were trypsinized, centrifuged and 1×106 cells re-suspended in 500 μl of FACS buffer and sorted using a FACSAria (BD Biosciences). Sorted cells were expanded by a further passage and checked by flow cytometry to ensure a pure population of transduced cells were maintained and used for downstream experiments.

Bioluminescent Imaging in a Bioreactor

Culture medium containing 150 μg/ml D-Luciferin was injected into the internal chamber of the bioreactor via the 3-way luer taps and imaged as described above. The bioreactor was placed on the stage and imaged. Stage D was used for zoomed out images of the entire reactor and stage C for all other images and analysis.

Other Materials

Megacell medium comprises 5% FBS, 1% Penicillin Streptomycin, 1% L-Glutamine, 1% non-essential amino acids, 0.1 mM Beta Mercapto Ethanol and 5 ng/ml Basic FGF.

For cryopreservation the media composition was 50% Fetal Bovine Serum (FBS), 40% MEGACELL Medium with supplements (described above), and 10% dimethyl sulfoxide (DMSO, Me2SO; Sigma, UK).

Slow cooling was achieved using “Mr Frosty” (Nalgene) freezing containers. Nalgene freezing containers were kept at −80° C. overnight.

### Example 2—Oesophagus

Rat decellularized oesophagi seeded with human mesoangioblasts (MABs), mouse fibroblasts (FBs) and mouse neural crest cells, were cultured in a bioreactor for up to 11 days and then frozen with the following protocol:
The seeded scaffold (size 7+20 mm length) was placed in a cryovial (size: 2 mL) with 500 μL FBS.
The vial was kept in ice throughout the process.
Another 500 μL were added of a solution of Megacell medium containing 20% DMSO.
The vial was transferred in a Nalgene freezing container and kept at −80° C. overnight.
Samples were then placed and stored in the vapour phase of liquid nitrogen at approximately −160° C.
After 2 to 4 weeks in the liquid nitrogen container, vials were rapidly thawed at 37° C. and samples transferred in 10-20 mL culture medium (Megacell supplemented with FBS and antibiotic) at 37° C. under mild agitation for 20 minutes.
Samples were then transferred to a culture petri dish with fresh culture medium and left in static culture for up to 7 days.

Cell viability and localization were confirmed with bioluminescence and histology. Scaffolds seeded with either MABs alone or co-seeded with MABs+FBs showed comparable survival after freezing, measuring radiance with IVIS before and after cryopreservation (FIG. 2A, FIG. 2B). Cell growth was clear immediately after thawing, with a continuous increase in bioluminescence emitted by seeded scaffolds throughout the 14 days of culture post-cryopreservation. At the end of the culture, scaffolds were analysed with histology to confirm cellular presence and in the correct localization of the oesophageal muscle layer. These results indicated not only cell survival after cryopreservation independently by the type of cell seeded, but also highlighted their capability to grow perfectly after thawing.

Cell differentiation and orientation within the scaffold was further assessed with immunostaining for hNuclei and GFP (FIG. 2C). Scaffolds co-seeded with a combination of MABs, FBs and GFP+ neural crest cells were cultured in dynamic conditions for 11 days, cryopreserved for 14 days, thawed and cultured for further 7 days. Immunofluorescence imaging showed how cell orientation, GFP+ neural crest cell morphology and cell-cell interaction were preserved after cryo-preservation, with comparable results to non-stored scaffolds.

### Example 3—Liver

Human decellularized liver cubes (5×5×5 mm) or human decellularized liver-derived Hydrogel cubes (5×5×5 mm) seeded with HepG2 cell line, cultured in static conditions for up to 10 days were frozen with the following protocol:
The seeded scaffold was placed in a cryovial (size: 2 mL) with 500 μL FBS.
The vial was kept in ice throughout the process.
Another 500 μL were added of a solution of alpha MEM containing 20% DMSO.
The vial was transferred in a Nalgene freezing container and kept at −80° C. overnight.
Samples were then placed and stored in the vapour phase of liquid nitrogen at approximately −160° C.
After 2 weeks in the liquid nitrogen container, vials were rapidly thaw at 37° C. and samples transferred in 5-10 mL culture medium (alpha MEM containing 10% FBS, 1% Antibiotic, 1% 1 mM sodium pyruvate, 1% non-essential AA solution 100×)) at 37° C. under mild agitation for 20 minutes.
Samples were then transferred to a culture petri dish with fresh culture medium and left in static culture for 3 days.

Subsequent analysis showed that the cells survived freezing and showed albumin production comparable with pre-freezing samples.

Albumin measurement: was performed using Abcam's Serum Albumin (ALB) in vitro SimpleStep ELISA® (Enzyme-Linked Immunosorbent Assay) kit. This is designed for the quantitative measurement of Serum Albumin protein in human serum and plasma.

The SimpleStep ELISA® employs an affinity tag labeled capture antibody and a reporter conjugated detector antibody which immunocapture the sample analyte in solution. This entire complex (capture antibody/analyte/detector antibody) is in turn immobilized via immunoaffinity of an anti-tag antibody coating the well. To perform the assay, samples or standards are added to the wells, followed by the antibody mix. After incubation, the wells are washed to remove unbound material. TMB substrate is added and during incubation is catalyzed by HRP, generating blue coloration. This reaction is then stopped by addition of Stop Solution completing any color change from blue to yellow. Signal is generated proportionally to the amount of bound analyte and the intensity is measured at 450 nm. Optionally, instead of the endpoint reading, development of TMB can be recorded kinetically at 600 nm.

### Example 4—Maintenance of Cell Viability after Storage

An experiment was designed to further demonstrate maintenance of cell viability following storage of a scaffold prepared and cryopreserved using the developed protocol.

Oesophageal scaffolds seeded with Luc⁺ZsGreen⁺MAB+FB were cultured in static conditions and then cryopreserved for 2 weeks in the same manner as described for Example 2. After storage, the samples were thawed and grown for 7 days in static culture. Cell viability was detected at various stages pre- and post-cryopreservation using bioluminescence (FIG. 4A, FIG. 4C) and an MTT assay (FIG. 4B).

After 7 days of culture the number of caspase 3 positive (caspase3⁺) cells was determined using immunofluorescence. Tissue samples were fixed in paraformaldehyde and frozen. 7-10 μm thick sections were cut with a cryostat and incubated with primary and secondary antibodies diluted in 1% Goat Serum/PBS/0.01% Triton X-100. Images were acquired with a Zeiss LSM 710 confocal microscope (Zeiss) and processed using ImageJ and Adobe Photoshop. Manual cell counting was performed to calculate the number of caspase3⁺ cells over the total number of DAPI⁺ cells in random sections from different regions of scaffolds.

The results demonstrate that the bio-engineered muscle that were cryopreserved with a slow-cooling process showed maintenance of cell viability after storage. Post-thawing, scaffolds showed a slight reduction in cell viability when compared to before cryopreservation. However, cells were able to recover and grow for up to 7 days in static culture, as confirmed by bioluminescence reading and MTT assay (FIG. 4A-FIG. 4C). Very few caspase3⁺ cells (<0.1%) were found in the cryopreserved scaffolds after 7 days of culture (data not shown).

### REFERENCES

1. Atala A. Engineering tissues, organs and cells. J Tissue Eng Regen Med 2007; 1:83-96. pmid:18038397 doi: 10.1002/term.18.
2. Orens J B, Garrity E R. General overview of lung transplantation and review of organ allocation. Proc Am Thorac Soc 2009; 6:13-9. doi: 10.1513/pats.200807-072GO. pmid:19131526.
3. Orlando G, Wood K J, De Coppi P, Baptista P M, Binder K W, Bitar K N, et al. Regenerative medicine as applied to general surgery. Ann Surg 2012; 255:867-80.
4. Maghsoudlou P, Urbani L, De Coppi P. Organ bioengineering for the newborn. Semin Pediatr Surg. 2014 October; 23(5):314-23.
5. Elliott M J, De Coppi P, Speggiorin S, Roebuck D, Butler C R, Samuel E, et al. Stem-cell-based, tissue engineered tracheal replacement in a child: a 2-year follow-up study. Lancet 2012; 380:994-1000. doi: 10.1016/S0140-6736(12)60737-5. pmid:22841419.
6. Totonelli G, Maghsoudlou P, Garriboli M, Riegler J, Orlando G, et al. (2012) A rat decellularized small bowel scaffold that preserves villus-crypt architecture for intestinal regeneration. Biomaterials 33: 3401-3410.
7. Totonelli G, Maghsoudlou P, Georgiades F, Garriboli M, Koshy K, et al. (2013) Detergent enzymatic treatment for the development of a natural acellular matrix for oesophageal regeneration. Pediatr Surg Int 29: 87-95.
8. Maghsoudlou P, Georgiades F, Tyraskis A, Totonelli G, Loukogeorgakis S P, et al. (2013) Preservation of micro-architecture and angiogenic potential in a pulmonary acellular matrix obtained using intermittent intra-tracheal flow of detergent enzymatic treatment. Biomaterials 34: 6638-6648.
9. Fishman J M, Lowdell M W, Urbani L et al. Immunomodulatory effect of a decellularized skeletal muscle scaffold in a discordant xenotransplantation model. Proceedings of the National Academy of Sciences of the United States of America, vol. 110, no. 35, pp. 14360-14365, 2013.
10. Badylak S F, Taylor D, Uygun K. Whole-Organ Tissue Engineering: Decellularization and Recellularization of Three-Dimensional Matrix Scaffolds. Annual Reviews; 2011 Jul. 14;
11. Maghsoudlou P, Totonelli G, Loukogeorgakis S P, Eaton S, De Coppi P. A decellularization methodology for the production of a natural acellular intestinal matrix. J Vis Exp. 2013 January; (80).
12. Totonelli G, Maghsoudlou P, Garriboli M, Riegler J, Orlando G, Burns A J, et al. A rat decellularized small bowel scaffold that preserves villus-crypt architecture for intestinal regeneration. Biomaterials. 2012 April; 33(12):3401-10.
13. [deleted]
14. Maghsoudlou P, Georgiades F, Tyraskis A, Totonelli G, Loukogeorgakis S P, Orlando G, et al. Preservation of micro-architecture and angiogenic potential in a pulmonary acellular matrix obtained using intermittent intra-tracheal flow of detergent enzymatic treatment. Biomaterials. 2013 September; 34(28):6638-48.
15. Piccoli M, Urbani L, Alvarez-Fallas M E, Franzin C, Dedja A, Bertin E, et al. Improvement of diaphragmatic performance through orthotopic application of decellularized extracellular matrix patch. Biomaterials. 2016 January; 74:245-55.
16. Hamilton N J, Kanani M, Roebuck D J, Hewitt R J, Cetto R, Culme-Seymour E J, Toll E, Bates A J, Cornerford A P, McLaren C A, Butler C R, Crowley C, McIntyre D, Sebire N J, Janes S M, O'Callaghan C, Mason C, De Coppi P, Lowdell M W, Elliott M J, Birchall M A. Tissue-Engineered Tracheal Replacement in a Child: A 4-Year Follow-Up Study. Am J Transplant. 2015 October; 15(10):2750-7.
17. KR 1020140037677 A
18. KR 1020140112465 A
19. WO 2015/151047 A1
20. EP 2885969 A1
21. WO 99/38952 A2

1. A method for the cryopreservation of a cellularised scaffold, which method comprises:
(i) providing a cellularised scaffold;
(ii) equilibrating said cellularised scaffold with a cryopreservative composition comprising culture medium and between 5 and 30% of a cryoprotectant;
(iii) freezing the equilibrated cellularised scaffold by reducing the temperature at between −0.8° C. and −1.2° C./minute to between −78° C. to −82° C.;
iv) storing the frozen cellularised scaffold at a temperature of between −135° C. and −198° C.

2. A method as claimed in claim 1 wherein step (iii) comprises freezing the equilibrated cellularised scaffold by continuously reducing the temperature at about −1° C./minute to about −80° C.

3. A method as claimed in claim 2 wherein step (i) comprises:
(ia) providing an acellular scaffold;
(ib) seeding the acellular scaffold with the cells;
(ic) culturing the seeded scaffold to produce said cellularised scaffold.

4. A method as claimed in claim 3 wherein step (ia) comprises:
(ia-1) providing a tissue or organ or sample thereof;
(ia-2) decellularizing said tissue or organ or sample thereof using one or both of detergents and enzymes to provide an acellular scaffold.

5. A method as claimed in claim 1 wherein the scaffold is a sheet scaffold, which is preferably tubular.

6. A method as claimed in claim 1 wherein the scaffold is derived from an organ or tissue which has been decellularized, wherein the organ is preferably luminal.

7. A method as claimed in claim 1 wherein the scaffold is of non-human origin.

8. A method as claimed in claim 1 wherein the cellularised scaffold is a tissue engineered oesophagus construct, which is optionally suitable for a neonate or infant.

9. A method as claimed in claim 7 wherein the cellularised scaffold is derived from decellularised oesophagus seeded with mesoangioblasts and fibroblasts.

10. A method as claimed in claim 9 wherein the culture medium is FBS plus Megacell medium comprising 1% Penicillin Streptomycin; 1% L-Glutamine; 1% non-essential amino acids; 0.1 mM Beta Mercapto Ethanol and 5 ng/ml Basic FGF.

11. A method as claimed in claim 1 wherein the scaffold is spherical, cuboid, cylindrical, hexagonal prismatic, conical, frustoconical, or pyramidal.

12. A method as claimed in claim 11 wherein the scaffold is cuboid.

13. A method as claimed in claim 1 wherein the scaffold is derived from a solid organ which has been decellularized.

14. A method as claimed in claim 11 wherein the cellularised scaffold is tissue engineered liver.

15. A method as claimed in claim 14 wherein the cellularised scaffold is a decellularised liver tissue seeded with human hepatic cells, which are optionally HepG2 cells.

16. A method as claimed in claim 1 wherein the scaffold is a hydrogel scaffold.

17. A method as claimed in claim 16 wherein the cellularised scaffold is a hydrogel scaffold seeded with human hepatic cells, which are optionally HepG2 cells.

18. A method as claimed in claim 17 wherein the culture medium comprises FBS plus a liver-cell supporting medium.

19. A method as claimed in claim 14 wherein the cellularised scaffold is liver model tissue for pharmacological research.

20. A method as claimed in claim 1 wherein the cellularised scaffold is tissue engineered lung, intestine, pancreas, muscle or bladder.

21. A method as claimed in claim 1 wherein the cryopreservative composition comprises 80% or more culture medium.

22. A method as claimed in claim 21 wherein the cryopreservative composition comprises between 5% to 15%, more preferably 8 to 12%, more preferably about 10% cryoprotectant.

23. A method as claimed in claim 1 wherein the cryoprotectant is selected from the list consisting of: dimethyl sulfoxide (DMSO); Ethylene glycol; Glycerol; 2-Methyl-2,4-pentanediol; Propylene glycol; Sucrose; Trehalose.

24. A method as claimed in claim 1 wherein step (ii) is carried out below ambient temperature, optionally at about 0 to 4° C.

25. A method as claimed in claim 1 wherein step (iv) is carried out by placing the equilibrated cellularised scaffold within one or more containers in the vapour phase of liquid nitrogen such as to achieve a temperature of about −160° C.

26. A method as claimed in claim 1 wherein step (iv) is carried out for at least 1, 2, 4, or 4 weeks.

27. A method as claimed in claim 1 further comprising:
(v) thawing the frozen cellularised scaffold rapidly in a water bath, optionally at 37° C.

28. A cryopreserved cellularised scaffold obtained according to the method of claim 1.

29. A thawed cryopreserved cellularised scaffold obtained according to the method of claim 27.

30. A kit comprising a cryopreserved cellularised scaffold of claim 28 and one or more containers.

31. A kit according to claim 30, wherein said kit further comprises instructions or labeling for the use of said kit for therapeutic purposes or for pharmacological research purposes.

32. A method of treatment of a subject with a chronic illness leading to organ failure, which method comprises:
(i) providing a cellularised scaffold which is a tissue engineered organ replacement using autologous cells of the subject;
(ii) cryopreserving the organ replacement according to the method of claim 1;
(iii) thawing the organ replacement when it is required by said subject;
(iv) treating said subject using said organ replacement.

33. A system for providing allogeneic tissue engineered organ replacements, which system comprises:
(i) providing a plurality cellularised scaffolds which are allogeneic tissue engineered organ replacements using universal donor iPS cells;
(ii) cryopreserving the organ replacements according to the method of claim 1;
(iii) identifying a subject in need to an organ replacement;
(iv) identifying a compatible cryopreserved allogeneic tissue engineered organ replacement;
(v) thawing the organ replacement when it is required by said subject;
(vi) treating said subject using said organ replacement.

34. A method for providing a three dimensional engineered micro-scaffold for use in pharmacological research purposes, which method comprises:
(i) providing a cellularised scaffold which is a three dimensional engineered micro-scaffold suitable for pharmacological research purposes;
(ii) cryopreserving the engineered micro-scaffold according to the method of claim 1;
(iii) thawing the micro-scaffold when it is required.

35. A cellularised scaffold or cryopreserved cellularised scaffold for use in the method or system of claim 32.

36. Use of a cellularised scaffold or cryopreserved cellularised scaffold for the preparation of a medical implant or material, for use in a method or system of claim 32.
